(19) United States
(12) Patent Application Publication
(10) Pub. No.: US 2002/0173845 A1
(43) Pub. Date: Nov. 21, 2002

(54) Low surface-glare intraocular lenses

(76) Inventors: Mark H. Bandhauer, Orange, CA (US); Jay C. Erie, Rochester, MN (US)

Correspondence Address: BAUSCH & LOMB INC., ONE BAUSCH & LOMB PLACE, ROCHESTER, NY, 14606-2701, US

(21) Appl. No.: 09/862,152

(22) Filed: May 21, 2001

(51) Int. Cl. A61F 2/16

(52) U.S. Cl. 623/6.17

(57) ABSTRACT

An intraocular lens with low surface-glare. The intraocular lens is constructed to preferably have a equi-biconvex optic form and a positive anterior optic surface radius of curvature less than 20 mm or greater than 33 mm. The intraocular lens of the present invention having the described construction minimizes or eliminates unwanted optical images and/or glare.

### FIELD OF THE INVENTION

The present invention relates to low surface-glare intraocular lenses and a method of making the same. More particularly, the present invention relates to intraocular lenses that minimize the amount of visible light reflected onto the retina of a patient from the anterior surface of the intraocular lens optic and a method of making the same. Intraocular lenses made in accordance with the present invention are particularly useful in aphakic eyes where a cataractous natural lens has been surgically removed.

### BACKGROUND OF THE INVENTION

A variety of intraocular lens (IOL) optic designs and materials are now commercially available to the cataract surgeon. Common optic designs include equi-biconvex and unequal biconvex. Common optic materials include silicone, acrylic and polymethylmethacrylate (PMMA). Although different optic designs and materials may have the same emmetropizing power, variations between differing optic designs and materials may form retinal images of differing quality. Clinical reports suggest that patients with acrylic IOL implants occasionally notice excessive glare and haloes around point sources of light and outside observers see external reflections from the IOLs. Although these symptoms are usually minimal, some patients have been so bothered by glare after implantation of an acrylic IOL that they requested and received an IOL exchange to eliminate the symptoms. While the absolute number of patients requiring IOL explantation is low, it has been found that glare or optical aberrations is a common reason for explantation of acrylic IOLs.

Undesirable optical effects attributed to IOLs have been referred to as glare, optical aberrations or unwanted optical images. Various optic shapes, optic diameters and optic edge designs have been reported as potential causes of such glare, optical aberrations or unwanted optical images. Because of the noted shortcomings of some IOL designs/materials with regard to glare, optical aberrations or unwanted optical images, there is a need for IOLs that minimize such undesirable optical effects.

### SUMMARY OF THE INVENTION

The present invention is a low surface-glare intraocular lens (IOL). The subject IOL comprises an optic portion for focusing visible light on the retina of a patient. The optic portion has an integral edge surface that defines the circumference of the optic portion. The optic portion likewise has opposed anterior and posterior surfaces adjacent the edge surface. The optic portion may be piano-convex, plano-concave, equi-biconvex, unequal biconvex or concave-convex depending upon the desired diopter of correction and the desired dimensions for efficient handling and implantation. An IOL made in accordance with the present invention has an optic portion with a positive anterior surface radius of curvature outside a range of 20 to 33 mm, regardless of diopter, to minimize optic anterior surface-glare. By maintaining a positive anterior surface radius of curvature outside of the specified range, undesirable optical effects are minimized or eliminated.

Accordingly, it is an object of the present invention to provide intraocular lenses for use in aphakic eyes.

Another object of the present invention is to provide intraocular lenses that minimize or eliminate undesirable optical effects.

Another object of the present invention is to provide intraocular lenses for use in aphakic eyes that minimize or eliminate undesirable optical effects.

Another object of the present invention is to provide intraocular lenses that minimize or eliminate surface-glare.

Still another object of the present invention is to provide intraocular lenses that minimize or eliminate optic anterior surface-glare.

These and other objectives and advantages of the present invention, some of which are specifically described and others that are not, will become apparent from the detailed description, drawings and claims that follow, wherein like features are designated by like numerals.

### BRIEF DESCRIPTION OF THE DRAWINGS

FIG. 1 is a schematic representation of the interior of an aphakic human eye including an intraocular lens implanted in the posterior chamber of the eye;

FIG. 2 is a plan view of an intraocular lens with two haptics made in accordance with the present invention;

FIG. 3 is a cross-sectional view along line 2-2 of the intraocular lens of FIG. 2 with an equi-convex optic portion;

FIG. 4 is a cross-sectional view along line 2-2 of the intraocular lens of FIG. 2 with an unequal-convex optic portion;

FIG. 5 is a cross-sectional view along line 2-2 of the intraocular lens of FIG. 2 with a plano-convex optic portion;

FIG. 6 is a cross-sectional view along line 2-2 of the intraocular lens of FIG. 2 with a piano-concave optic portion;

FIG. 7 is a cross-sectional view along line 2-2 of the intraocular lens of FIG. 2 with a concave-convex optic portion;

FIG. 8 is a diagram of the external reflectivity of a prior art silicone intraocular lens;

FIG. 9 is a diagram of the external reflectivity of a prior art acrylic intraocular lens;

FIG. 10 is a diagram of the internal reflectivity of a prior art silicone intraocular lens; and

FIG. 11 is a diagram of the internal reflectivity of a prior art acrylic intraocular lens.

### DETAILED DESCRIPTION OF THE INVENTION

FIG. 1 illustrates a simplified diagram of an eye 10 showing landmark structures relevant to the implantation of an intraocular lens (IOL) of the present invention. Eye 10 includes an optically clear cornea 12, and an iris 14. A lens capsule 16 and a retina 18 are located behind the iris 14 of eye 10. Eye 10 also includes anterior chamber 20 located in front of iris 14 and a posterior chamber 22 located between iris 14 and lens capsule 16. An IOL 24, such as that of the present invention, is preferably implanted in lens capsule 16 after the natural lens (not shown) has been removed therefrom (aphakic application). Eye 10 also includes an optical axis OA-OA that is an imaginary line that passes through the optical center 21 of eye 10. Optical axis OA-OA in human eye 10 is generally perpendicular to a portion of cornea 12, lens capsule 16 and retina 18.

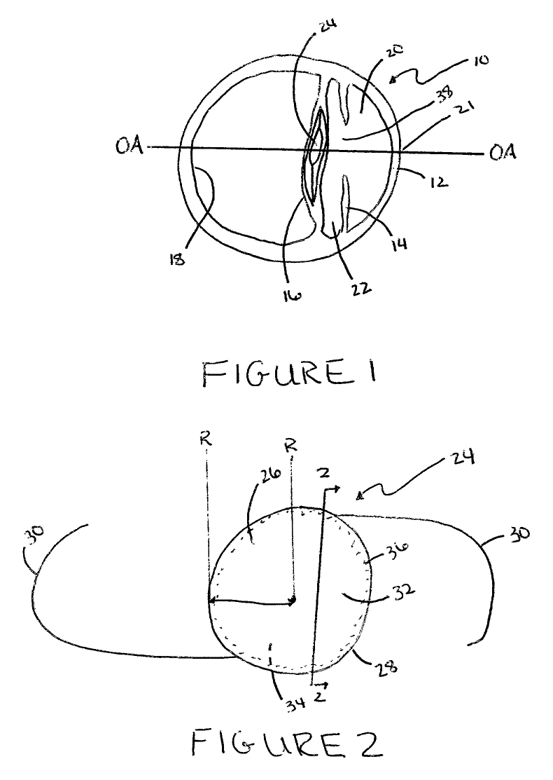

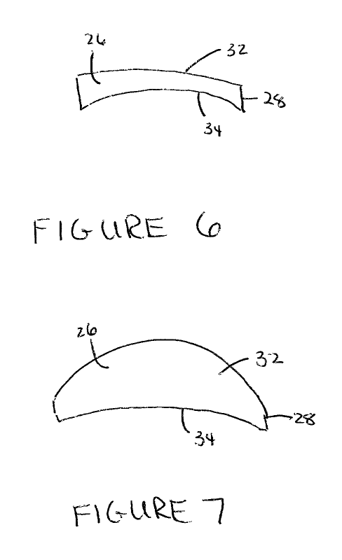

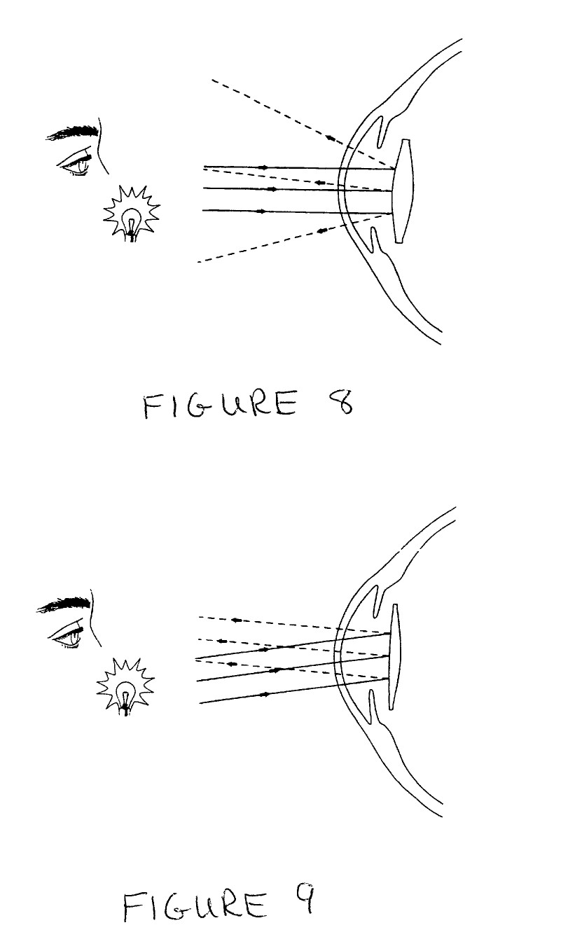

The IOL of the present invention, illustrated in FIGS. 1 through 7 identified by reference numeral 24, is designed for implantation preferably in lens capsule 16 of a patient's eye 10. However, IOL 24 may likewise be implanted in other suitable locations within eye 10 as known to those skilled in the art of ophthalmic surgery. IOL 24 has an optic portion 26 with an outer peripheral edge 28. Preferably integrally formed on peripheral edge 28 of optic portion 26 are one or more haptic support elements 30. Alternatively however, one or more haptic support elements 30 may be attached to optic portion 26 by staking, chemical polymerization or other methods known to those skilled in the art of IOL manufacture. Haptic support elements 30 maintain IOL 24 in a position within eye 10 perpendicular to optical axis OA-OA.

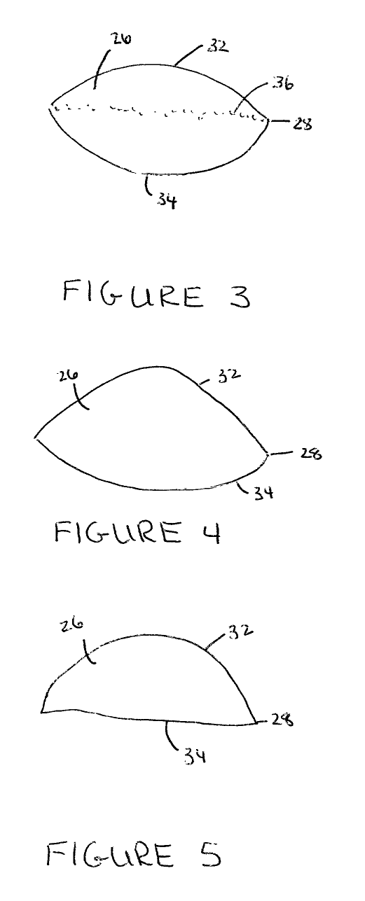

Optic portion 26 of IOL 24 includes a peripheral edge 28 and opposed anterior and posterior surfaces, 32 and 34 respectively. As illustrated in FIGS. 3 through 7, optic portion 26 may have any of a number of different forms including equi-biconvex, unequal biconvex, plano-concave, plano-convex and concave-convex depending upon the desired diopter of correction and the desired dimensions. The dimensions of the subject IOL are dictated by the requirements of efficient handling and implantation, preferably through a small surgical incision of approximately 3.2 mm or smaller. Regardless of the specific form of optic portion 26, in accordance with the present invention, the positive radius of curvature R-R of anterior surface 32 must be outside of the “glare range” of 20 mm to 33 mm. By having the positive radius of curvature R-R of anterior surface 32 outside of the glare range, undesirable optic effects such as surface glare, optical aberrations and/or unwanted optical images are minimized or eliminated.

The mechanism for undesirable optic effects such as surface glare, optical aberrations and/or unwanted optical images may be explained as follows. In an eye with a prior art intraocular lens implant having a positive anterior surface radius of curvature within the specified glare range of 20 mm to 33 mm, an incoming light beam which is relatively collimated passes through the intraocular lens to the retina. However, a portion of the incoming light beam is retroreflected by the anterior surface of the intraocular lens into an outgoing collimated beam. This outgoing collimated beam is visible to an outside observer and the internally reflected retinal image, or glare, is visible to the patient. The mechanism for undesirable optic effects is described in still greater detail below.

Prior Art Intraocular Lens Optical Analysis:

A study was conducted including a lens manufactured of a lower refractive index (RI) silicone, RI=1.43, having an equi-biconvex optic design with a positive anterior and posterior radius of curvature of 10.0 mm, a lens manufactured of polymethylmethacrylate (PMMA), RI=1.49, having an equi-biconvex optic design with a positive anterior and posterior radius of curvature of 15.0 mm and a lens manufactured of a higher refractive index acrylic material, RI=1.55, having an unequal biconvex optic design with a positive 32.0 mm anterior and a positive 16.0 mm posterior radius of curvature.

The formation of glare images from an external light source by an IOL was evaluated by modeling the light source, the eye, and the IOL using the Zemax™ optical design program (Focus Software, Inc.). The external light source consisted of collimated light at 2.5 degrees or 12.5 degrees to the optical axis. A physiologic eye model was designed with the following physiological parameters: corneal power, 38.0, 40.0, 43.0 and 46.0 diopters (D); anterior chamber depth, 4.5 mm (posterior surface of the cornea to anterior surface of the IOL); approximate axial length, 23.5 mm; IOL power, 20.0 D; optic diameter, 6.0 mm, and pupil diameter, 3.5 mm. For each IOL model studied, the optical design program traced rays from the light source through the pseudophakic eye model to construct an externally reflected image visible to an outside observer at a distance of 1 m and an internally reflected retinal image visible to the patient.

To evaluate externally reflected light, the optical design program traced rays from the light source, through the cornea, and then to the convex anterior surface of the IOL. The program then traced reflected rays from the anterior surface of the IOL back to the outside observer as illustrated in FIGS. 8 and 9, wherein hatched lines represent reflected light rays. As illustrated in FIG. 8, an equi-biconvex optic design with a steep anterior radius of curvature of 10.0 mm, acts as a strong convex mirror. Reflected light rays leaving the pseudophakic eye are divergent and little if any light would enter the observer's pupil. As illustrated in FIG. 9, an unequal biconvex optic design with a flat anterior radius of curvature of 32.0 mm acts as a weak convex mirror, allowing the convergent wavefront transmitted by the cornea to be at near normal incidence to the anterior surface of the IOL. Subsequently, reflected light leaving the pseudophakic eye is nearly collimated or minimally divergent. Thus, more light enters the observer's pupil than with the equi-biconvex design of FIG. 8. The externally reflected light from an IOL visible to an outside observer has been referred to as a “glint” or “flash”.

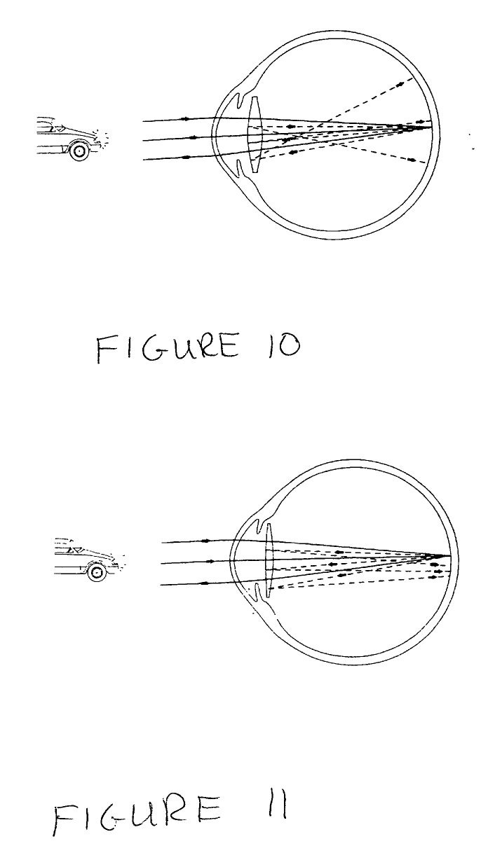

To evaluate externally reflected light, the optical design program traced rays from the light source, through the IOL, and to the retina. It is known that the human fundus acts as a diffuse reflector and as much as 75% of light focused on the fundus is reflected anteriorly. Fundus reflectivity is the basis for ophthalmoscopy. Therefore, the program traced anteriorly reflected rays from the fundus back to the concave anterior surface of the IOL, where they were reflected back to the retina to form a glare spot of measurable area as illustrated in FIGS. 10 and 11. FIG. 10 illustrates a light source at 2.5 degrees from the visual axis producing a refracted and focused image on the retina depicted as solid lines. Anteriorly reflected light from the fundus depicted as hatched lines are redirected posteriorly by a second reflection from the anterior surface of the IOL to form a second retinal glare image which is defocused, round and large, i.e., approximately 34.0 mm². FIG. 11 illustrates a light source at 2.5 degrees from the visual axis producing a refracted and focussed image on the retina depicted as solid lines. Anteriorly reflected light from the fundus depicted as hatched lines are redirected posteriorly by a second reflection from the anterior surface of the IOL to form a second retinal glare image which is focused, round and small, i.e., approximately 0.56 mm².

The refractive index of the IOL material and the design of the IOL optic contribute to reflection and the subsequent glare perceived by the retina or by the outside observer. Reflectivity at the anterior surface of the IOL was evaluated using classical optical surface analysis. As light passes through a boundary, part of the incident light is reflected and part is refracted and transmitted. Light incident from the side of the rarer medium (aqueous, RI=1.336) is external reflection and light incident from the side of the denser medium is internal reflection. The reflectivity (r) of an optical material can be estimated by combining Fresnel's reflectivity equations and Snell's law to obtain

$$r = {( \frac{{n}_{2} cos B - {n}_{1} cos a}{{n}_{2} cos B + {n}_{1} cos a} )}^{2}$$

where a is the angle of incidence and B is the angle of refraction. Reflectivity increases as the difference between refractive indices and/or the angle of incidence, a, increases. At normal incidence, a=B=0 and reflectivity, r, becomes

$$r = {( \frac{{n}_{2} - {n}_{1}}{{n}_{2} + {n}_{1}} )}^{2}$$

Substantial reflection effects resulting from light rays will be shown at very close to normal incidence so that the above equation may be applied.

The size and brightness of the internally reflected retinal glare image was evaluated using the Zemax software program. For each optic design and corneal power studied, the software program determined the area in square mm of the defocused reflected image at the curved retinal surface. The relative intensity, or brightness, of the retinal glare image for each IOL model was described and compared in terms of a relative intensity ratio proportional to reflectivity (%)/area (mm²). The relative intensity ratio of the silicone lens with a corneal power of 43.0 D and incident light at 2.5 degrees to optical axis, was arbitrarily designated as 1.

The intensity of the externally reflected glare image was determined by calculating the area of the reflected external image at a distance of 1 m from the IOL. The area was ratioed to the area of a typical observer's pupil to obtain a relative area value. No relative area less than 1 was allowed to eliminate the possible exaggeration of the effect if the entire glare only filled a portion of the observer's pupil. A typical area of 8 mm²for a 3.5 mm pupil was used. The ratio was then multiplied by relative reflectivity (r/r_{silicone}) to obtain the relative intensity ratio for external glare. The relative intensity ratio of the silicone lens with a corneal power of 43.0 D was arbitrarily designated as 1.

The above-described study showed that light reflected from the anterior surface of an IOL increased as the refractive index of the IOL material increased. In the human eye, silicone with a RI=1.43, PMMA with a RI=1.49 and acrylic with a RI=1.55 optic materials reflected approximately 0.11%, 0.30% and 0.55% of light at normal incidence, respectively. The use of a higher refractive index material with any optic design increased reflected light 5-fold compared to a lower refractive index material.

With regard to internal reflection, the above-described study showed that external rays at 2.5 and 12.5 degrees to the optical axis were reflected anteriorly from the fundus and then redirected posteriorly toward the retina from a second reflection off the anterior surface of the IOL. The silicone equi-biconvex optic design produced an internally reflected glare image that was defocused to a large area as illustrated in FIG. 10. In contrast, the acrylic unequal biconvex optic design with the flatter anterior radius of curvature produced a glare image that was focused to a small area as illustrated in FIG. 11. Combining an unequal biconvex optic design with higher refractive index acrylic increased the relative intensity of reflected light at the retina 300-fold compared to the equi-biconvex optic design composed of lower refractive index silicone. With regard to external reflection, combining an unequal biconvex optic design with higher refractive index acrylic increased the relative intensity of external reflected light 400-fold compared to the equi-biconvex optic design composed of lower refractive index silicone.

Based upon the findings of the above-described study, the low surface-glare IOLs 24 of the present invention are preferably of a equi-biconvex optic portion 26 design to minimize or eliminate surface-reflected glare and unwanted optical images. An equi-biconvex optic 26 design with a steep anterior surface radius of curvature R-R of less than 20 mm causes internally reflected light from IOL 24 to pass through a focus far enough in front of retina 18 to reduce the intensity on retina 18 and the potential for unwanted optical images. This is true regardless of the refractive index of the material comprising optic portion 26. An equi-biconvex optic portion 26 designs with a flatter anterior surface 32 radius of curvature R-R of greater than 33 mm were too steep to allow the converging wavefront passing through cornea 12 to approach normal incidence at anterior surface 32 to reduce or eliminate the potential for unwanted optical images. This is true regardless of the refractive index of the material comprising optic portion 26. However, preferably the subject IOLs 24 are manufactured using a silicone, PMMA or acrylic material having a refractive index of 1.55 or less.

Optic portion 26 of IOL 24 is a positive powered lens from 0 to approximately +40 diopters. Optic portion 26 may be biconvex, plano-convex, piano-concave, or concave-convex (meniscus) but most preferably equi-biconvex.

Optic portion 26 of the subject IOL 24 may optionally be formed with a glare reduction zone 36 of approximately 0.25 to 0.75 mm but more preferably approximately 0.3 to 0.6 mm and most preferably 0.5 mm in width adjacent outer peripheral edge 28 for reducing glare when outer peripheral edge 28 of IOL 24 is struck by light entering eye 10 during high light or at other times when pupil 38 is dilated. Glare reduction zone 36 is typically fabricated of the same material as optic portion 26, but may be opaque, colored or patterned in a conventional manner to block or diffuse light in plane with optical axis OA-OA.

Subject IOL 24 is preferably manufactured by first producing discs from a material of choice as described in U.S. Pat. Nos. 5,217,491 and 5,326,506 each incorporated herein in its entirety by reference. IOL 24 may then be machined from the material discs in a conventional manner. Once machined, IOL 24 may be polished, cleaned, sterilized and packaged by a conventional method known to those skilled in the art. Alternatively, IOL 24 may be molded in accordance with methods known to those skilled in the art of intraocular lens manufacture.

Subject IOL 24 is used in eye 10 by creating an incision in cornea 12, inserting IOL 24 in either anterior chamber 20 or posterior chamber 22 and closing the incision in accordance with methods known to those skilled in the art. Alternatively, IOL 24 may be used in eye 10 by creating an incision in cornea 12 and lens capsule 16, removing the natural lens from lens capsule 16, inserting IOL 24 in lens capsule 16 and closing the incision in accordance with methods known to those skilled in the art.

While there is shown and described herein certain specific embodiments of the present invention, it will be manifest to those skilled in the art that various modifications may be made without departing from the spirit and scope of the underlying inventive concept and that the same is not limited to the particular forms herein shown and described except insofar as indicated by the scope of the appended claims.

We claim:

1. An acrylic intraocular lens to be implanted within an eye generally perpendicular to the eye's optical axis comprising:
an outer peripheral edge defining an optic portion;
an anterior optic surface with a positive radius of curvature less than 20 mm or greater than 33 mm;
a posterior optic surface; and
one or more haptic support elements permanently connected or formed on the outer peripheral edge.

2. An intraocular lens to be implanted within an eye generally perpendicular to the eye's optical axis comprising:
an outer peripheral edge defining an optic portion manufactured from a material having a refractive index of 1.50 or greater;
an anterior optic surface with a positive radius of curvature less than 20 mm or greater than 33 mm;
a posterior optic surface; and
one or more haptic support elements permanently connected or formed on the outer peripheral edge.

3. An intraocular lens to be implanted within an eye generally perpendicular to the eye's optical axis comprising:
an outer peripheral edge defining an optic portion manufactured to have an equi-biconvex form from a material having a refractive index of 1.50 or greater;
an anterior optic surface with a positive radius of curvature less than 20 mm or greater than 33 mm;
a posterior optic surface; and
one or more haptic support elements permanently connected or formed on the outer peripheral edge.

4. The intraocular lens of claim 2 or 3 wherein said lens is formed from an acrylic, silicone or polymethylmethacrylate material.

5. The intraocular lens of claim 1 wherein said lens is formed from an acrylic material having a refractive index greater than 1.50.

6. The intraocular lens of claim 1, 2 or 3 wherein a glare reduction zone is formed adjacent to the outer peripheral edge of the optic portion.

7. A method of manufacturing the intraocular lens of claim 1, 2 or 3 comprising:
forming a disk of a suitable material; and
machining said lens from said disk.

8. A method of manufacturing the intraocular lens of claim 1, 2 or 3 comprising:
injecting a suitable material in a mold; and
curing said material prior to removal from said mold.

9. A method of using the intraocular lens of claim 1, 2 or 3 comprising:
creating an incision in a cornea of an eye; and
inserting said intraocular lens in an anterior chamber of said eye.

10. A method of using the intraocular lens of claim 1, 2 or 3 comprising:
creating an incision in a cornea of an eye; and
inserting said intraocular lens in a posterior chamber of said eye.

11. A method of using the intraocular lens of claim 1, 2 or 3 comprising:
creating an incision in a cornea and lens capsule of an eye;
removing a natural lens of said eye from said lens capsule; and
inserting said intraocular lens in said lens capsule of said eye.
